(19) United States
(12) Patent Application Publication
(10) Pub. No.: US 2007/0196323 A1
(43) Pub. Date: Aug. 23, 2007

(54) Polyvinyl alcohol-containing compositions and methods for dermal delivery of drugs

(76) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,134

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/750,521, filed on Dec. 14, 2005.
(60) Provisional application No. 60/577,536, filed on Jun. 7, 2004.

(51) Int. Cl. A61K 31/74

(52) U.S. Cl. 424/78.02

(57) ABSTRACT

The present invention is drawn to adhesive solidifying formulations, and methods for dermal delivery of a drug. The formulation can include a drug, a solvent vehicle, and a polyvinyl alcohol. The solvent vehicle can include a volatile solvent system including water and an alcohol solvent, e.g., ethanol, propanol, and/or isopropanol, and a non-volatile solvent system including at least one non-volatile solvent which is compatible with polyvinyl alcohol. The formulation is formulated such that the water to polyvinyl alcohol weight ratio is in the range of from about 4:1 to about 1:1, and water to alcohol solvent weight ratio in the range of from about 0.33:1 to about 6:1.

This application claims the benefit of U.S. Provisional Application Nos. 60/750,637 and 60/750,521, each of which was filed on Dec. 14, 2005, and is a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to formulations developed for dermal delivery of drugs. More particularly, the present invention relates to adhesive solidifying formulations having a viscosity suitable for application to a skin surface, and which form a sustained drug-delivering adhesive solidified layer on the skin.

### BACKGROUND OF THE INVENTION

Traditional dermal drug delivery systems can generally be classified into two forms: semisolid formulations and dermal patch dosage forms. Semisolid formulations are available in a few different forms, including ointments, creams, foams, pastes, gels, or lotions and are applied topically to the skin. Dermal (including transdermal) patch dosage forms also are available in a few different forms, including matrix patch configurations and liquid reservoir patch configurations. In a matrix patch, the active drug is mixed in an adhesive that is coated on a backing film. The drug-laced adhesive layer is typically directly applied onto the skin and serves both as means for affixing the patch to the skin and as a reservoir or vehicle for facilitating delivery of the drug. Conversely, in a liquid reservoir patch, the drug is typically incorporated into a solvent system which is held by a thin bag, which can be a thin flexible container. The thin bag can include a permeable or semi-permeable membrane surface that is coated with an adhesive for affixing the membrane to the skin. The membrane is often referred to as a rate limiting membrane (although it may not actually be rate limiting in the delivery process in all cases) and can control transport of the drug from within the thin bag to the skin for dermal delivery.

While patches and semisolid formulations are widely used to deliver drugs into and through the skin, they both have significant limitations. For example, most semisolid formulations usually contain only volatile solvent(s), such as water and ethanol, which evaporate shortly after application. The evaporation of such solvents can cause a significant decrease or even termination of dermal drug delivery, which may not be desirable in many cases. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing. Drugs present in a semisolid formulation may also be unintentionally delivered to persons who come in contact with a subject undergoing treatment with a topical semisolid formulation.

With respect to matrix patches, in order to be delivered appropriately, a drug should have sufficient solubility in the adhesive, as primarily only dissolved drug contributes to the driving force required for skin permeation. Unfortunately, solubility in adhesives that is too low does not generate adequate skin permeation driving force over sustained period of time. In addition, many ingredients, e.g., liquid solvents and permeation enhancers, which could be used to help dissolve the drug or increase the skin permeability, may not be able to be incorporated into many adhesive matrix systems in sufficient quantities to be effective. For example, at functional levels, most of these materials may adversely alter the wear properties of the adhesive. As such, the selection and allowable quantities of additives, enhancers, excipients, or the like in adhesive-based matrix patches can be limited. To illustrate, for many drugs, optimal transdermal flux can be achieved when the drug is dissolved in certain liquid solvent systems, but a thin layer of adhesive in a typical matrix patch often cannot hold enough appropriate drug and/or additives to be therapeutically effective. Further, the properties of the adhesives, such as coherence and tackiness, can also be significantly changed by the presence of liquid solvents or enhancers.

Regarding liquid reservoir patches, even if a drug is compatible with a particular liquid or semisolid solvent system carried by the thin bag of the patch, the solvent system still has to be compatible to the adhesive layer coated on the permeable or semi-permeable membrane; otherwise the drug may be adversely affected by the adhesive layer or the drug/solvent system may reduce the tackiness of the adhesive layer. In addition to these dosage form considerations, reservoir patches are bulkier and usually are more expensive to manufacture than matrix patches.

Another shortcoming of dermal (including transdermal) patches is that they are usually neither stretchable nor flexible, as the backing film (in matrix patches) and the thin fluid bag (in reservoir patches) are typically made of polyethylene or polyester, both of which are relatively non-stretchable materials. If the patch is applied to a skin area that is significantly stretched during body movements, such as a joint, separation between the patch and skin may occur thereby compromising the delivery of the drug. In addition, a patch present on a skin surface may hinder the expansion of the skin during body movements and cause discomfort. For these additional reasons, patches are not ideal dosage forms for skin areas subject to expansion, flexing and stretching during body movements.

In view of the shortcomings of many of the current delivery systems, it would be desirable to provide systems, formulations, and/or methods that can i) provide sustained drug delivery over long periods of time; ii) are not vulnerable to unintentional removal by contact with clothing, other objects, or people for the duration of the application time; iii) can be applied to a skin area subject to stretching and expansion without causing discomfort or poor contact to skin; and/or iv) can be easily removed after application and use.

### SUMMARY OF THE INVENTION

It has been recognized that it would be advantageous to provide dermal delivery formulations, systems, and/or methods in the form of adhesive solidifying compositions or formulations having a viscosity suitable for application to the skin surface, and which form a drug-delivering solidified layer on the skin that can optionally be easily peelable or removable by washing or other approach after use.

In accordance with this, a formulation for dermal delivery of a drug can comprise a drug, polyvinyl alcohol, and a solvent vehicle. The solvent vehicle can include a volatile solvent system comprising water and an alcohol solvent, and a non-volatile solvent system comprising at least one non-volatile solvent that is less volatile than water. The non-volatile solvent system can facilitate transdermal delivery of the drug at a therapeutically effective rate over a sustained period of time. The water to polyvinyl alcohol weight ratio can be from about 4:1 to about 1: 1, and the water to alcohol solvent weight ratio can be from about 0.33:1 to about 5:1. In one particular embodiment, the molecular weight of the polyvinyl alcohol can be from 10,000 to 100,000 MW.

In an alternative embodiment, a method of dermally delivering a drug can comprise applying a formulation to a skin surface of a subject. The formulation can comprise a drug; a polyvinyl alcohol; and a solvent vehicle which includes a volatile solvent system including water and an alcohol solvent. Additionally, the solvent vehicle also comprises a non-volatile solvent system of at least one non-volatile solvent that is less volatile than water. The non-volatile solvent system can facilitate dermal delivery of the drug at a therapeutically effective rate over a sustained period of time. The water to polyvinyl alcohol weight ratio can be from about 4:1 to about 1:1, and the water to alcohol solvent weight ratio can be from about 0.33:1 to about 6:1. Additional steps include solidifying the formulation to a solidified layer on the skin surface by at least partial evaporation of the volatile solvent system, and dermally delivering the drug from the solidified layer to the subject at therapeutically effective rates over a sustained period of time.

Additional features and advantages of the invention will be apparent from the following detailed description which illustrates, by way of example, features of the invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

The term “drug(s)” refers to any bioactive agent that is applied to, into, or through the skin which is applied for achieving a therapeutic affect. This includes compositions that are traditionally identified as drugs, as well other bioactive agents that are not always considered to be “drugs” in the classic sense, e.g., peroxides, humectants, emollients, etc., but which can provide a therapeutic effect for certain conditions. When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced herein can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic sufficient levels of the drug across, onto or into the subject's skin when the non-volatile solvent system is saturated with the drug. For systemically targeted drugs, a flux enabling non-volatile solvent system is a non-volatile solvent system that can provide therapeutically sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” or “therapeutically sufficient flux” is defined as the permeation flux of the selected drug that delivers sufficient amount of drug into or across the skin to be clinically beneficial. “Clinically beneficial” or “clinically sufficient” when referring to flux means at some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400 cm²/10 hour, which equals 1 mcg/cm²/hr. By this definition, different drugs have different “therapeutically effective flux.” Therapeutically sufficient flux” may be different in different subjects and or at different times for even the same subject. However, for each drug, there is usually a consensus among the skilled in the art on the range of doses or fluxes that are sufficient in most subjects at most times.

The following are estimates of flux for some drugs that are therapeutically effective or more than sufficient:

TABLE A
In vitro steady state flux values of various drugs

| | | Estimated |
|---|---|---|
| | | Therapeutically |
| | | sufficient flux* |
| Drug | Indication | (mcg/cm²/h) |
| Ropivacaine** | Neuropathic pain | 5 |
| Lidocaine | Neuropathic pain | 30 |
| Acyclovir | Herpes simplex virus | 3 |
| Ketoprofen | Musculoskeletal pain | 16 |
| Diclofenac | Musculoskeletal pain | 1 |
| Clobetasol | Dermatitis, psoriasis, | 0.05 |
| | eczema | |
| Betamethasone | Dermatitis, psoriasis, | 0.01 |
| | eczema | |
| Testosterone | Hypogonadal men, | 0.8 [ |
| Testosterone | Hormone treatment for | 0.25 |
| | postmenopausal women | |
| Imiquimod | Warts, basal cell | 0.92 |
| | carcinoma | |

*Flux determined using an in vitro method described in Example 1.
**Estimated flux based on known potency relative to lidocaine.

The therapeutically effective flux values in Table A (with the exception of ropivacaine) represent the steady state flux values of marketed products through hairless mouse or human epidermal membrane in an in vitro system described in Example 1. These values are meant only to be estimates and to provide a basis of comparison for formulation development and optimization. The therapeutically effective flux for a selected drug could be very different for different diseases to be treated for, different stages of diseases, and different individual subjects. It should be noted that the flux listed may be more than therapeutically effective.

The following examples listed in Table B illustrate screening of non-volatile solvent's flux enabling ability for some of the drugs specifically studied. Experiments were carried out as described in Example 1 below and the results are further discussed in the subsequent Examples 2-9.

TABLE B
In vitro steady state flux values of various drugs from non-volatile
solvent systems

| | | Average Flux* |
|---|---|---|
| Drug | Non-Volatile Solvent | (mcg/cm²/hr) |
| Betamethasone | Oleic acid | 0.009 ± 0.003 |
| Dipropionate | Sorbitan Monolaurate | 0.03 ± 0.02 |
| Clobetasol Propionate | Propylene Glycol (PG) | 0.0038 ± 0.0004 |
| | Light Mineral Oil | 0.031 ± 0.003 |
| | Isostearic acid (ISA) | 0.019 ± 0.003 |
| Ropivacaine | Glycerol | 1.2 ± 0.7 |
| | Mineral Oil | 8.9 ± 0.6 |
| Ketoprofen | Polyethylene glycol 400 | 5 ± 2 |
| | Span 20 | 15 ± 3 |
| Acyclovir | Polyethylene glycol 400 | 0 |
| | Isostearic acid + 10% | 2.7 ± 0.6 |
| | trolamine | |

*Each value represents the mean and st. dev. of three determinations.

The in vitro steady state flux values in Table B from non-volatile solvents show surprising flux-enabling and non flux-enabling solvents. This information can be used to guide formulation development.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

Different drugs often have different matching flux-enabling non-volatile solvent systems which provide particularly good results. Examples of such are noted in Table C. Experiments were carried out as described in Example 1 below and the results are further discussed in the subsequent Examples 2-9.

TABLE C
In vitro steady state flux values of various drugs from particularly high
flux-enabling non-volatile solvent systems

| | High flux-enabling non- | Avg. Flux* |
|---|---|---|
| Drug | volatile solvent | (mcg/cm²/h) |
| ropivacaine | ISA | 11 ± 2 |
| | Span 20 | 26 ± 8 |
| ketoprofen | Propylene glycol (PG) | 90 ± 50 |
| acycolvir | ISA + 30% trolamine | 7 ± 2 |
| Betamethasone | Propylene Glycol | 0.20 ± 0.07 |
| Dipropionate | | |
| Clobetasol | PG + ISA (Ratio of PG:ISA | 0.8 ± 0.2 |
| propionate | ranging from 200:1 to 1:1) | |

*Each value represents the mean and st. dev of three determinations.

It should be noted that “flux-enabling non-volatile solvent,” “flux-enabling, plasticizing non-volatile solvent,” or “high flux-enabling non-volatile solvent” can be a single chemical substance or a mixture of two or more chemical substances. For example, the steady state flux value for clobetasol propionate in Table C is a 9:1 for propylene glycol:isostearic acid mixture that generated much higher clobetasol flux than propylene glycol or ISA alone (see Table B). Therefore, the 9:1 propylene glycol:isostearic acid mixture is a “high flux-enabling non-volatile solvent” but propylene glycol or isostearic acid alone is not.

The term “adhesion” or “adhesive” when referring to a solidified layer herein refers to sufficient adhesion between the solidified layer and the skin so that the layer does not fall off the skin during intended use on most subjects. Thus, the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the current invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

The term “adhesive” in relation to the solidified layer means it is adhesive to the skin on which the original formulation was applied, and not necessarily, and preferably not, adhesive on the other side to other objects.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention, it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), various aliphatic resins and aromatic resins, or combinations thereof.

The terms “washable” or “removed by washing” etc., when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol, or combinations thereof. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or at least partially dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

“Molecular weight” or “average molecular weight” when used in relation to polyvinyl alcohol in the current invention means weight average molecular weight.

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, a formulation for dermal delivery of a drug can comprise a drug, polyvinyl alcohol, and a solvent vehicle. The solvent vehicle can include a volatile solvent system including water and an alcohol solvent, and a non-volatile solvent system, including at least one non-volatile solvent that is less volatile than water. The non-volatile solvent system can facilitate dermal delivery of the drug at a therapeutically effective rate over a sustained period of time. The water to polyvinyl alcohol weight ratio can be from about 4:1 to about 1:1, and the water to alcohol solvent weight ratio can be from about 0.33:1 to about 6:1.

In an alternative embodiment, a method of dermally delivering a drug can comprise applying a formulation to a skin surface of a subject. The formulation can comprise a drug; a polyvinyl alcohol; and a solvent vehicle which includes a volatile solvent system including water and an alcohol solvent. Additionally, the solvent vehicle also includes a non-volatile solvent system of at least one non-volatile solvent that is less volatile than water. The non-volatile solvent system can facilitate dermal delivery of the drug at a therapeutically effective rate over a sustained period of time. The water to polyvinyl alcohol weight ratio can be from about 4:1 to about 1:1, and the water to alcohol solvent weight ratio can be from about 0.33:1 to about 6:1. Additional steps include solidifying the formulation to form a solidified layer on the skin surface by at least partial evaporation of the volatile solvent system, and dermally delivering the drug from the solidified layer to the subject at therapeutically effective rates over a sustained period of time.

In each of the above embodiments, i.e. the formulation and method, the water to polyvinyl alcohol weight ratio can range from about 4:1 to about 1:1, and water to alcohol weight ratio can range from about 0.33:1 to about 6:1. In still another aspect, the water to polyvinyl alcohol weight ratio can range from about 3:1 to about 1.2:1, or from about 2:1 to about 1.4:1, and the weight ratio of water to alcohol solvent can be from about 0.75:1 to about 6:1, or from about 0.75:1 to about 2:1.

Thus, the present invention is related to formulations that are typically semi-solids (including creams, gels, pastes, ointments, and other viscous liquids) in the initial form, which can be easily applied onto the skin as a layer, and can quickly (from 15 seconds to 4 minutes under standard skin and ambient conditions) to moderately quickly (from 4 to 15 minutes under standard skin and ambient conditions) change into a solidified layer, e.g., a coherent and soft solid layer that is optionally peelable, for drug delivery. A solidified layer thus formed is capable of delivering drug to the skin, into the skin, across the skin, etc., at therapeutically effective rates, over a sustained period of time, e.g., hours to tens of hours, so that most of the drug delivery occurs after the solidified layer is formed. Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus capable of maintaining good contact with a skin surface, even if the skin is stretched during body movement, such as at a knee, finger, elbow, or other joints.

Solidifying formulations comprising polyvinyl alcohol (PVA) as the solidifying agent in the present invention, may optionally also contain appropriate amounts of other auxiliary solidifying agent(s). In one aspect of the present invention, the polyvinyl alcohol has a weight average molecular weight of at least 10,000. In addition the solidifying formulations can contain amounts of non-volatile solvent(s) that are compatible with polyvinyl alcohol, such as glycerol and propylene glycol, which when combined together can yield formulations that form solidified layer with many desirable properties such as: elasticity, high stretchability, good tensile strength, and/or adherence to the skin surface where the formulation is applied on but exhibits minimal tack to other skin surfaces or objects. However, selecting or formulating a volatile solvent system for such a formulation that is compatible with all other ingredients and allows the formulation layer applied on skin to “dry” within desirable time has been challenging. Water can be used as the volatile solvent for such formulations. However, formulations made with water alone take quite long time to form a non-tacky surface after being applied on the skin surface, typically more than 10 minutes under standard testing conditions. Water may also be incompatible with other ingredients such as auxiliary solidifying agents that are needed to achieve certain desirable properties. For example, Dermacryl 79 (National Starch) can be added to increase adhesion to the skin, but it is not soluble in water. Therefore, a solvent that is more volatile and/or a solvent that is more compatible with the ingredients such as auxiliary solidifying agents would be desirable. This being said, though water evaporates from solidifying formulations slowly, to the knowledge of the inventors, polyvinyl is substantially insoluble in any volatile solvents other than water. Thus, when the above-listed volatile solvents are used without water, the formulations exhibit solid lumps and very poor homogeneity that renders them unusable.

On the other hand, when polyvinyl alcohol in water solution is mixed with an appropriate amount of a non-volatile solvent, optionally also with an appropriate type and/or amount of an auxiliary solidifying agent(s), and the resulting solution is cast on skin and let dry, one of two things may form on the skin, depending on the compatibility of the non-volatile solvent with polyvinyl alcohol. If the non-volatile solvent is compatible with polyvinyl alcohol, a homogeneous, flexible, solidified layer is formed, and little to no liquid oozes out of the solidified layer. If the non-volatile solvent is not compatible with polyvinyl alcohol, the resulting matter on skin will not be homogeneous and most likely some liquid will ooze out of the matter. Therefore, a non-volatile solvent is defined as being compatible with polyvinyl alcohol if a formulation containing polyvinyl alcohol, water, and the said non-volatile solvent can form a homogeneous layer after being cast on the skin as a thin layer and after water has evaporated off. Examples of non-volatile solvents that are compatible with polyvinyl alcohol include without limitation glycerol, isopropanol, propylene glycol, oleic acid, olelyl alcohol, poly ethylene glycol, isostearic acid, and Span 20 (sorbitan monolaurate).

Through many experiments, it has been discovered that solutions with a relatively narrow range of water to ethanol ratio can serve as good volatile solvent for solidifying formulations comprising polyvinyl alcohol and appropriate non-volatile solvents. For example, polyvinyl alcohol (MW 31,000-50,000), glycerol, water, and ethanol, in 2:2:3:3 weight ratio can be formulated into a clear, homogeneous, smooth, and viscous liquid that can be easily applied to a skin surface as a thin layer, and which can form a non-tacky surface on human skin under normal conditions much faster than a formulation that has the same components but no water. In this formulation, polyvinyl alcohol is the solidifying agent, glycerol is the non-volatile solvent system, and a 3:3 water/ethanol solution is the volatile solvent system. This formulation dries much faster than similar formulations using only water as the volatile solvent. This approach can solve the dilemma that water as a volatile solvent causes slow drying but water is still needed for polyvinyl alcohol to function.

In one embodiment, it has also been discovered that the ratio of polyvinyl alcohol, water, and ethanol can be particularly effective in a relatively narrow range in order for the formulations to have both the desired drying time and homogeneity. In order to have more desirable homogeneity, the water to polyvinyl alcohol weight ratio typically can be at least about 3:1, to about 1.2:1. In another embodiment, the water to polyvinyl alcohol ratio can be about 2:1 to about 1.5:1. When the water to polyvinyl alcohol weight ratio is greater than 4:1, the drying time can be longer than desirable. If the water to ethanol weight ratio ranges from 1:2 to 1:3, the polyvinyl alcohol can become less compatible with the solvent mixture and the formulations tend to have hard lumps and less desirable homogeneity. When the water to ethanol weight ratio is increased and is in the range of about 2:1 to 3:1, the drying time becomes significantly longer. Therefore, in one embodiment, a solidifying formulation comprising polyvinyl alcohol as the solidifying agent and appropriate non-volatile solvent(s), the ratio of water to polyvinyl alcohol can be in the range from about 3:1 to about 1.2:1, or in another aspect from about 2:1 to about 1.5:1. Similarly, the water to ethanol ratio should be in the range of about 0.5:1 to about 5:1, or in another aspect from about 0.75:1 to about 1.5:1. Since propanol and isopropanol have similar properties as ethanol, they can substitute for ethanol in the aforementioned formulations and ratios. A volatile solvent system including ethanol, propanol, isopropanol, or a combination thereof, is considered to be a preferred “alcohol solvent” herein. For formulations that also contain auxiliary solidifying agent(s), the weight of polyvinyl alcohol in above ratios can be replaced by the total weight of polyvinyl alcohol plus all other solid auxiliary solidifying agents.

As mentioned briefly above, it is to be noted that the formulations comprising a drug, a polyvinyl alcohol, a non-volatile solvent system, water and alcohol solvent may also contain other ingredients such additional auxiliary solidifying agents. Examples of additional solidifying agents include but are not limited to polyethylene oxide, ammonia methacrylate, carrageenan, cellulose acetate phthalate aqueous such as CAPNF from Eastman, carboxy methyl cellulose Na, carboxy polymethylene, cellulose, cellulose acetate (microcrystalline), cellulose polymers, divinyl benzene styrene, ethyl cellulose, ethylene vinyl acetate, silicone, polyisobutylene, shellac (FMC BioPolymer), guar gum, guar rosin, cellulose derivatives such as hydroxy ethyl cellulose, hydroxy methyl cellulose, hydroxy propyl cellulose, hydroxypropyl methyl cellulose, carboxymethyl cellulose, and methyl cellulose, hypromellose phthalate (hydroxypropyl methylcellulose phthalate), methyl acrylate, microcrystalline wax, polyvinyl acetate, polyvinyl acetate phthalate such as Suretic from Colorcon, PVP ethyl cellulose, polyvinyl pyrrolidone (PVP), acrylate, PEG/PVP, xantham Gum, trimethyl siloxysilicate, maleic acid/anhydride copolymers, polacrilin, poloxamer, polyethylene oxide, poly glactic acid/poly-I-lactic acid, turpene resin, locust bean gum, prolamine (Zein), acrylic copolymers, polyurethane dispersions, gelatin, dextrin, starch, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid) copolymers and methylmethacrylate copolymers, including Rohm and Haas' Eudragit polymers (Eudragit (E, L, NE, RL, RS, S100)), Esters of polyvinylmethylether/maleic anhydride copolymer such as Gantrez ES-425, Gantrez ES-225 available from ISP, ethyl cellulose, Dermacryl polymers (National Starch), certain Eudragit polymers (Rohm & Haas), and combinations thereof.

The non-volatile solvent system and the solidifying agent are desirably compatible with each other. Compatibility can be defined as i) the solidifying agent does not substantially negatively influence the function of the non-volatile solvent system, except for some reduction of flux; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent (including PVA and any auxiliary solidifying agents) can be from about 0.1:1 to about 10:1. In another aspect, the ratio between the non-volatile solvent system and the solidifying agent can be from about 0.5:1 to about 2:1.

Irrespective of the non-volatile solvents mentioned above, other examples of non-volatile solvents which can be used in the solidifying formulations of the present invention include but are not limited to 1,2,6-gexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzoic acid, benzyl alcohol, butyl alcohol, Bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, dibutyl subecate, diethanolamine, dietthylene glycol monoethyl ether, diglycerides, dipropylene glycol, ethylene glycol, eucalyptus oil, eugenol, rose oil, fat, fatty acid (esters glycerides), fatty alcohols, liquid sugars, ginger extract, glycerin, high fructose corn syrup, hydrogenated castor oil, IPM, IP palmitate, isostearic acid, lemon oil, lime oil, limonene, milk, mineral oil, monoacetin, monoglycerides, nutmeg oil, oleic acid, octyldodecanol, oleyl alcohol, olive alcohol, orange oil, palm oil, polyethylene glycol PEG, peanut oil, PEG, vegetable oil, peppermint oil, petrolatum, phenol, pine needle oil, polypropylene glycol, propylene glycol, sesame oil, spearmint oil, soybean oil, trolamine, tromethemine, vegetable oil, vegetable shortening, vinyl acetate, vitamin E, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters including PEG stearates, PEG oleates, PEG-laurates, PEG fatty acid diesters including PEG-dioleates, PEG-distearates PEG-castor oils, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid multisterol extract, myristyl alcohol, neutral oil, PEG octyl phenyl ether, PEG-alkyl ethers including PEG-cetyl ethers, PEG-stearyl ethers, PEG-sorbitan fatty acid esters including PEG-sorbitan diisosterates, PEG-sorbitan monostearates, propylene glycol fatty acid esters such as propylene glycol stearates, propylene glycol caprylate/caprate, sodium pyrrolidone carboxylate, sorbitol, squalene, stear-o-wet, triacetin, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty alcohol ethers, alkyl-amides (N,N-dimethylalkylamides), N-methyl pyrrolidone and related compounds, sorbitan fatty acid surfactants such as sorbitan monooleate, sorbitan trioleate, sorbitan monopalmitate, ethyl oleate, polyglycerized fatty acids, glycerol, glycerol monooleate, glyceryl monomyristate, glycerol esters of fatty acids, isopropyl myristate, or combinations thereof.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface as the solidified layer is forming. If the drug is very potent and the solidified layer has very high tensile strength, a layer as thin as 0.01 mm may be sufficient. If the drug has rather low potency and/or the solidified layer has low tensile strength, a layer as thick as 2-3 mm may be desirable. Thus, for most drugs and formulations, the appropriate thickness can be from about 0.01 mm to about 3 mm, but more typically, from about 0.05 mm to about 1 mm.

The flexibility and stretchability of the solidified layer, which is optionally peelable, can be desirable in some applications. For instance, certain non-steroidal anti-inflammatory agents (NSAIDs) can be applied directly over joints and muscles for transdermal delivery into joints and muscles. However, skin areas over joints and certain muscle groups are often significantly stretched during body movements. Such movement prevents non-stretchable patches from maintaining good skin contact. Lotions, ointments, creams, gels, foams, pastes, or the like also may not be suitable for use for the reasons cited above. As such, in transdermal delivery of NSAIDs into joints and/or muscles, the solidifying formulations of the present invention can offer unique advantages and benefits.

A further feature of a formulation prepared in accordance with embodiments of the present invention is related to drying time. If a formulation dries too quickly, the user may not have sufficient time to spread the formulation into a thin layer on the skin surface before the formulation is solidified, leading to poor skin contact. If the formulation dries too slowly, the user may have to wait a long time before resuming normal activities (e.g. putting clothing on) that may remove un-solidified formulation. Thus, it is desirable for the drying time to be longer than about 15 seconds but shorter than about 15 minutes, and preferably from about 0.5 minutes to about 4 minutes.

Other benefits of the solidified layers of the present invention include the presence of a physical barrier that can be formed by the material itself. For instance, local anesthetic agents and other agents such as clonidine may be delivered topically for treating pain related to neuropathy, such as diabetic neuropathic pain. Since many of such subjects feel tremendous pain, even when their skin area is touched with minimal pressure, the physical barrier of the solidified layer can prevent or minimize pain caused by accidental contact with objects or others.

These and other advantage can be summarized in the following non-limiting list of benefits, as follows. The solidified layers of the present invention can be prepared in an initial form that is easy to apply as a semisolid dosage form. Additionally, upon volatile solvent system evaporation, the dosage form is relatively thick and can contain much more active drug than a typical layer of traditional cream, gel, lotion, ointment, paste, etc., and further, is not as subject to unintentional removal. Further, as the solidified layer remains adhesive and is peelable, easy removal of the solidified layer can occur, usually without the aid of a solvent or surfactant. In some embodiments, the adhesion to skin and elasticity of the material is such that the solidified layer will not separate from the skin upon skin stretching at highly stretchable skin areas, such as over joints and muscles. For example, in one embodiment, the solidified layer can be stretched by 5%, or even 10% or greater, in one direction without cracking, breaking, and/or separating form a skin surface to which the layer is applied. Still further, the solidified layer can be formulated to advantageously deliver drug and protect sensitive skin areas without cracking or breaking. Additionally, the solidified layer can be peeled from the skin as a single or just a few large pieces relative to the total size of application (this property is referred to as “peelable), or washed from the skin using water or other solvent (depending on the formulation).

The solidifying formulations of the present invention can be formulated to treat a variety of conditions and disease such as musculoskeletal pain, neuropathic pain, alopecia, skin disease including dermatitis and psoriasis as well as skin restoration (cosmetic skin treatment), and infections including viral, bacterial, and fungal infection. As such the formulations can deliver a wide ranging number and types of drugs and active agents. In one embodiment the solidifying formulation can be formulated to include acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, or combinations thereof In one embodiment, the formulation can include an antifungal drug such as amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, and undecylenate, or combinations thereof.

In another embodiment, the formulation can include an antifungal drug such as acyclovir, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, or combinations thereof.

When the formulation is intended to provide antibacterial treatment it can be formulated to include an antibacterial drug such as erythromycin, clindamycin, tetracycline, bacitracin, neomycin, mupirocin, polymyxin B, quinolones such as ciproflaxin, or combinations thereof.

When the formulation is intended to relieve pain, particularly neuropathic pain, the formulation can include a local anesthetic such as lidocaine, bupivacaine, ropivacaine, and tetracaine; an alpha-2 agonists such as clonidine. When the formulation is intended to treat pain associated with inflammation it can be formulated to include an non-steroidal anti-inflammatory drug such as ketoprofen, piroxicam, diclofenac, indomethacin, COX inhibitors general COX inhibitors, COX-2 selective inhibitors, COX-3 selective inhibitors, or combinations thereof. Conversely, a patch containing a local anesthetic agent, such as Lidoderm™, is widely used for treating neuropathic pain, such as pain caused by post-herpetic neuralgia and diabetes induced neuropathic pain. Due to the limitations of the patch as discussed above, the solidified layers prepared in accordance with the present invention provide some unique benefits, as well as provide a potentially less expensive alternative to the use of a patch. Possible drugs delivered for such applications include, but are not limited to, local anesthetics such as lidocaine, prilocaine, tetracaine, bupivicaine, etidocaine; and other drugs including capsaicin and alpha-2 agonists such as clonidine, dissociative anesthetics such as ketamine, tricyclic antidepressants such as amitriptyline.

In another embodiment, the formulation can be formulated to treat skin disorders or blemishes by including active agents such as anti-acne drugs such as clindamycin and benzoyl peroxide, retinol, vitamin A derivatives such as tazarotene and isotretinoin, cyclosporin, anthralin, vitamin D3, cholecalciferol, calcitriol, calcipotriol, tacalcitol, calcipotriene, etc.

In yet another embodiment, the delivery of medication for treating warts and other skin conditions would also benefit from long periods of sustained drug delivery. Examples of anti-wart compounds include but are not limited to: imiquimod, rosiquimod, keratolytic agents: salicylic acid, alpha hydroxy acids, sulfur, rescorcinol, urea, benzoyl peroxide, allantoin, tretinoin, trichloroacetic acid, lactic acid, benzoic acid, or combinations thereof.

A further embodiment involves the use of the solidifying formulations for the delivery of sex steroids including but not limited to progestagens consisting of progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, androgens consisting of testosterone, methyl testosterone, oxandrolone, androstenedione, dihydrotestosterone. estrogens consisting of estradiol, ethniyl estradiol, estiol, estrone, conjugated estrogens, esterified estrogens, estropipate or combinations thereof.

Non-sex steroids can also be delivered using the formulations of the present invention. Examples of such steroids include but are not limited to betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, or combinations thereof.

A further embodiment involves controlled delivery of nicotine for treating nicotine dependence among smokers and persons addicted to nicotine. Formulations of the present invention would be a cost effective way of delivering therapeutic amounts of nicotine transdermally.

Another embodiment involves using the formulation to deliver anti-histamine agents such as diphenhydramine and tripelennamine. These agents would reduce itching by blocking the histamine that causes the itch and also provide relief by providing topical analgesia.

Other drugs which can be delivered using the solidifying formulations of the present invention include but are not limited to tricyclic anti-depressants such as amitriptyline; anticonvulsants such as carbamazepine and alprazolam; N-methyl-D-aspartate (NMDA) antagonists such as ketamine; 5-HT2A receptor antagonists such as ketanserin; and immune modulators such as tacrolimus and picrolimus.

Other drugs that can be used include humectants, emollients, and other skin care compounds.

Another feature of the formulations of the present invention is related to solidifying formulations comprising a drug, a non-volatile solvent system comprising at least one non-volatile solvent, a solidifying agent, and a volatile solvent system comprising a volatile solvent whose boiling point is below 20° C. (such a solvent can be used as a propellant or can be dissolved in the formulation). In one embodiment, the formulation can be stored in a pressurized container and be sprayed on the skin surface with the help of the propellant. Some hydrofluorocarbons commonly used as propellants in pharmaceutical or dosmetic industries can work in this design. More specifically, the propellants may include, but not limited to dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof. The formulation may also be expelled out of the container and applied on the skin via a manual pump. Formulations comprising these room temperature gaseous volatile solvents are expected to dry much faster. Spraying the formulation onto the skin suffering from neuropathic pain can avoid touching the skin with an applicator which can cause severe pain in the sometimes hypersensitive skin.

The formulations of the current invention may further comprise a pH modifying agent for adjusting the pH of the formulation to a point or a range most suitable for the delivery of the drug. This feature can be important for a drug that is ionizable.

As a further note, it is a unique feature of the solidified layers of the present invention that they can keep a substantial amount of the non-volatile solvent system, which is optimized for delivering the drug, on the body surface. This feature can provide unique advantages over existing products. For example, in some semi-solid formulations, upon application to a skin surface the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membranes as noted for the in vitro flux studies described in herein. Hairless mouse skin (HMS) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations (of Examples 2-5) on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Example 2

An adhesive formulation containing 0.05% (w/w) clobetasol propionate with propylene glycol and isostearic acid as non volatile solutions as well as plasticizers, and polyvinyl alcohol (MW 31,000-50,000) as a solidifying agent is prepared. The formulation is prepared from the ingredients as shown in Table 1.

TABLE 1
Solidifying formulation components

| | | | | Percent | Percent | |
|---|---|---|---|---|---|---|
| Ex- | | Percent | Percent | Propylene | Isostearic | Percent |
| ample | Polymer | Polymer | Ethanol | Glycol | Acid | Water |
| 2 | Polyvinyl | 20 | 30 | 19.6 | 0.4 | 30 |
| | Alcohol | | | | | |

The composition shown above is studied for flux of clobetasol propionate as shown in Table 2 as follows:

TABLE 2
Steady state flux of clobetasol propionate through human
cadaver skin at 35° C.

| | Skin Flux* |
|---|---|
| Formulation | (ng/cm²/h) |
| Example 2 | 87.8 ± 21.4 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

As seen from Table 2 the formulation described in Example 2 that contains polyvinyl alcohol as solidifying agent has high flux of clobetasol propionate. Polyvinyl alcohol forms stretchable solidified layers and it is likely that this formulation will have acceptable wear properties. The toughness of the resulting solidified layer can be modified by adding appropriate plasticizers if needed. Tackiness can also be modified by adding appropriate amounts of tackifier or by adding appropriate amounts of another solidifying agent such as acrylates/octylacrylamide copolymer Dermacryl 79

### Example 3

Prototype solidifying formulations are prepared as follows. Several solidified formulations are prepared in accordance with embodiments of the present invention in accordance with Table 3, as follows:

TABLE 3

Example 3
% by weight

Volatile Solvents

| | |
|---|---|
| Ethanol | 21 |
| Water | 32 |

Solidifying agents

| | |
|---|---|
| Polyvinyl Alcohol | 21 |
| (MW 31,000-50,000) | |

Non-volatile solvents/plasticizer

| | |
|---|---|
| Propylene Glycol | 21 |

Drug

| | |
|---|---|
| Ketoprofen | 5 |

Solidifying formulations of Examples 3 are prepared in the following manner:
The solidifying agents are dissolved in the volatile solvent (e.g., dissolve polyvinyl alcohol in water, Eudragit polymers in ethanol),
The non-volatile solvent is mixed with the solidifying agent/volatile solvent mixture.
The resulting solution is vigorously mixed well for several minutes.
The drug is then added and the solidifying formulation is mixed again for several minutes.

In all the Examples noted above, the flux-enabling non-volatile solvent/solidifying agent/volatile solvent combination is compatible as evidenced by a homogeneous, single phase system that exhibited appropriate drying time, and provided a stretchable solidified layer and steady state flux for the drug (see Example 4 below).

### Example 4

The formulation of Example 3 is tested in a hairless mouse skin (HMS) or HEM in vitro model described in Example 1. Table 4 shows data obtained using the experimental process outlined above.

TABLE 4
Steady-state flux (J)

| | J* |
|---|---|
| Formulation | (μg/cm²/h) |
| Example 3 | 35 ± 20** |

*Skin flux measurements represent the mean and standard deviation of three determinations.
**Flux measurements reported were determined from the linear region of the cumulative amount versus time plots. The linear region was observed to be between 4-8 hours. If experimental conditions allowed, the steady-state delivery would likely continue well beyond 8 hours.

Ketoprofen has lower steady state flux values when the enabling non-volatile solvent is incorporated into the solidifying formulation. This could be the result of the volatile solvent system or solidifying agent having the opposite impact on the chemical environment (e.g., decreasing solubility, physical interactions between drug and solidifying formulation) resulting in lower flux values.

### Example 5

A formulation with the following composition: 10.4% polyvinyl alcohol, 10.4% polyethylene glycol 400, 10.4% polyvinyl pyrrolidone K-90, 10.4% glycerol, 27.1% water, and 31.3% ethanol was applied onto a human skin surface at an elbow joint and a finger joint, resulting in a thin, transparent, flexible, and stretchable peel. After a few minutes of evaporation of the volatile solvents (ethanol and water), a solidified layer that was peelable was formed. The stretchable peel had good adhesion to the skin and did not separate from the skin on joints when bent, and could easily be peeled away from the skin.

### Examples 6-8

Three formulations are applied on the stratum corneum side of freshly separated hairless mouse skin. The in vitro flux is determined for each formulation as outlined in Example 1. The formulation compositions and the flux are noted in Table 5 below.

TABLE 5

| Example | | |
|---|---|---|
| 6 | 7 | 8 |

% by weight

| | | | |
|---|---|---|---|
| PVA (MW 31,000-50,000) | 15 | 15 | 15 |
| Water | 23 | 23 | 23 |
| Ethylcellulose N-100 | 11 | 11 | 11 |
| Ethanol | 33 | 33 | 33 |
| Span 20 | 11 | | |
| Polyethylene Glycol 400 | | 11 | |
| Tween 40 | | | 11 |
| Tromethamine | 4 | 4 | 4 |
| Ropivacaine HCl | 3 | 3 | 3 |
| Avg. Flux* (mcg/cm²/h) | 15 ± 1 | 4.7 ± 0.3 | 3.4 ± 0.7 |

*Flux values represent the mean and standard deviation of three determinations. Flux measurements reported were determined from the linear region of the cumulative amount versus time plots. The linear region was observed to be between 6-31 hours. If the experiment was continued it is anticipated the steady state would continue.

Examples 9-10

Two solidifying formulations for dermal delivery of imiquimod are prepared which both include a specified amount of imiquimod in an excipient mixture to form an adhesive formulation in accordance with embodiments of the present invention. The peel formulations contain the following components:

TABLE 6
Imiquimod peelable formulation ingredients

Example

| Ingredients* | 9 | 10 |
|---|---|---|
| PVA (mw 31,000-50,000) | 12 | 21.5 |
| Pemulen TR-2 | 0.3 | 0.3 |
| Water | 62.7 | 34.4 |
| Isopropanol | | |
| ISA (Isostearic Acid) | 19 | 35.2 |
| Trolamine | 2 | 3.6 |
| Imiquimod | 4 | 5 |

*Ingredients are noted as weight percent.
** Polymer from Degussa

These formulations are applied to HMS skin as described in Example 1, and the imiquimod flux is measured. A summary of the results from in vitro flux studies carried out with the formulations in Examples 9-10 are listed in Table 7.

TABLE 7
Steady-state flux of imiquimod through hairless mouse skin
from various adhesive formulations at 35° C.

| | Average flux | |
|---|---|---|
| Formulation | mcg/cm²/h* | Ratio to Control** |
| Example 9 | 0.7 ± 0.09 | 0.7 |
| Example 10 | 0.52 ± 0.06 | 0.6 |
| Aldara (control) | 0.92 ± 0.02 | |

*The flux values represent the mean and SD of three determinations
**Ratio to control calculated by dividing the flux value for each Example by the flux value for Aldara control flux.

Regarding the formulation described in Examples 9 and 10, water is used as the volatile solvent, and the ISA, trolamine mixture is used as the non-volatile solvent system and plasticizer. Through experimentation, it is determined that ISA and Span 20 provide the appropriate solubility for the drug, however, these non-volatile solvents are hydrophobic and not compatible with the volatile solvent system used to dissolve the solidifying agent (polyvinyl alcohol). An emulsifier Pemulen TR-2 was used to emulsify the non-volatile solvents into the water phase. Further, in this embodiment, ISA and trolamine act as a plasticizer in the peelable formulation after the water (volatile solvent) has evaporated. The steady state flux of formulation Examples 9 and 10 demonstrate the importance of the amount of non-volatile solvent in added to the formulation in dictating the flux-generating power of the entire formulation.

### Example 11

A solidifying formulation for dermal delivery of imiquimod is prepared which includes a specified amount of imiquimod in an excipient mixture to form an adhesive formulation in accordance with embodiments of the present invention. The peel formulations contained the following components:

TABLE 8
Imiquimod peelable formulation ingredients

| | Example |
|---|---|
| Ingredients* | 11 |
| PVA (mw 31,000-50,000) | 10.1 |
| Pemulen TR-2 | 0.3 |
| Water | 52.9 |
| ISA (Isostearic Acid) | 16.8 |
| Salicylic Acid | 15.2 |
| Trolamine | 1.7 |
| Triacetin | |
| Imiquimod | 3.0 |

*Ingredients are noted as weight percent.
** Polymer from Degussa

The formulation is applied to HMS skin as described in Example 1, and the imiquimod flux is measured. A summary of the results from in vitro flux studies carried out with the formulation of Example 11 is listed in Table 9.

TABLE 9
Steady-state flux of Imiquimod through hairless mouse skin
from various adhesive formulations at 35° C.

| | Average flux | Ratio to |
|---|---|---|
| Formulation | mcg/cm²/h* | Control** |
| Example 12 | 1 ± 1 | 1.1 |
| Aldara | 0.9 ± 0.02 | 1 |

*The flux values represent the mean and SD of three determinations
**Ratio to control calculated by dividing the flux value for each Example by the flux value for Aldara control flux.

Addition of a keratolytic agent (salicylic acid) slightly improves the permeability of imiquimod compared to Examples 10-11 and may be beneficial as a combination treatment for warts.

### Example 12

To demonstrate the ability of the solidified solidifying formulations to reduce the transepidermal water loss (TEWL) the following experiment was conducted.

Placebo polyvinyl alcohol formulation similar to the formulation described in Example 3 was applied to the top of the hand and the TEWL was measured on a site immediately adjacent to the solidified layer and on top of the solidified layer. The TEWL measurement of the site covered by the solidified layer was 33% lower than the untreated skin site.

Placebo neutral copolymer of butyl methacrylate and methyl methacrylate (Plastoid B) formulation similar to the formulation described in Example 6 was applied to the top of the hand and the TEWL was measured on a side immediately adjacent to the solidified layer and on top of the solidified layer. The TEWL measurement on the site covered by the solidified layer was 30% lower than the untreated skin site.

### Example 13

A solidifying formulation for dermal delivery of lidocaine is prepared which includes a saturated amount of lidocaine in an excipient mixture to form an adhesive peelable formulation in accordance with embodiments of the present invention. The solidified formulation is prepared from the ingredients as shown in Table 10.

TABLE 10
Lidocaine solidifying formulation components

| Ingredients* | Example 13 |
|---|---|
| PVA (mw 31,000-50,000) | 11.7 |
| Eudgragit E-100** | 11.7 |
| PVP-K90 | 5.8 |
| Glycerol | 8.8 |
| PEG-400 | 8.8 |
| Water | 23.8 |
| Ethanol | 23.8 |
| Lidocaine | 5.6 |

*Ingredients are noted as weight percent.
**from Rohm & Haas.

TABLE 11
Steady-state flux of Lidocaine through hairless mouse skin
from various adhesive solidifying formulations at 35° C.

| | Average flux |
|---|---|
| Formulation | mcg/cm²/h* |
| Example 13 | 47 ± 3 |

The adhesive formulation of lidocaine in the present example has similar physical properties to the formulations in the other examples noted above. The transdermal flux across hairless mouse skin is acceptable and steady-state delivery is maintained over 8 hours.

### Example 14

This solidifying formulation has the following ingredients in the indicated weight parts:

TABLE 12

| | | Ethyl | Dermacryl | | | | |
|---|---|---|---|---|---|---|---|
| | | Cellulose | 79 | | Isostearic | | |
| | | N-7 | (National | | Acid | | |
| PVA | Water | (Aqualon) | Starch) | Ethanol | (ISA) | Glycerol | Ropivacaine |
| 1 | 1.5 | 0.25 | 0.35 | 0.85 | 0.8 | 0.35 | 0.3 |

In this formulation, polyvinyl alcohol (USP grade, MW 31,000-50,000, from Amresco) is a solidifying agent, ethyl cellulose and Dermacryl 79 are auxiliary solidifying agents. Isostearic acid and glycerol form the non-volatile solvent system/plasticizer while ethanol and water form the volatile solvent system. Ropivacaine is the drug.

Procedures of making the formulation:
1. Ropvicaine is mixed with ISA.
2. Ethyl cellulose and Dermacryl 79 are dissolved in ethanol.
3. PVA is dissolved in water at temperature of about 60-70° C.
4. All of the above mixtures are combined together in one container and glycerol is added and the whole mixture is mixed well.
   The resulting formulation is a viscous fluid. When a layer of about 0.1 mm thick is applied on skin, a non-tacky surface is formed within about 2 minutes.

### Example 15

A solidifying formulation was prepared in accordance with Table 13, as follows:

TABLE 13
Peel-forming formulation for sex steroids

| Ingredient | % by weight |
|---|---|
| Ethanol | 43 |
| Water | 22 |
| Polyvinyl Alcohol (mw 31,000-50,000) | 14 |
| Glycerol | 14 |
| Polyethylene glycol | 6 |
| Testosterone | 1 |

The ingredients of Table 13 were combined as follows:
The solidifying agent is dissolved in the volatile solvent (i.e. dissolve polyvinyl alcohol in water).
The flux enabling non-volatile solvent is mixed with the solidifying agent/volatile solvent mixture.
The resulting solution is vigorously mixed well for several minutes.
Drug is then added and the peel formulation is mixed again for several minutes.

### Example 16

The formulation prepared in Example 15 was tested for Skin Flux, as set forth in Table 14 below.

TABLE 14
Peel-forming formulation for sex steroids

| | Skin Flux* |
|---|---|
| System | (mcg/cm²/h) |
| Example 15 | 4 ± 1 |
| AndroGel | 6 ± 2 |
| *Skin flux measurements represent the mean and standard deviation of three determinations. Flux measurements reported were determined from the linear region of the cumulative amount versus time plots. The linear region was observed to be between 4-8 hours. | |
| # If experimental conditions allowed, the steady-state delivery would likely continue well beyond 8 hours. | |

It should be noted, the steady-state flux value reported in Table 3 is determined using the linear region between 2-6 hours. The solidifying formulation in Example 15 will deliver a steady-state amount of testosterone for at least 9 hours.

### Examples 17-20

A stretchable adhesive formulation for transdermal delivery of ketoprofen (which is suitable for regional delivery via skin for treating inflammation or pain of joints and muscles) is prepared which includes saturated amount of ketoprofen in an excipient mixture (more ketoprofen than that can be dissolved in the excipient mixture) to form an adhesive formulation, some of which is prepared in accordance with embodiments of the present invention. The excipient mixture, which is a viscous and transparent fluid, is prepared using the ingredients as shown in Table 15.

TABLE 15
Ketoprofen solidifying formulation components

| | Examples | | | |
|---|---|---|---|---|
| Ingredients* | 17 | 18 | 19 | 20 |
| PVA (Polyvinyl Alcohol) | 10.4 | 21.4 | 21.1 | 21.2 |
| (mw 31,000-50,000) | | | | |
| PEG-400 (Polyethylene | 10.4 | 10.8 | 2.9 | 18.6 |
| Glycol) | | | | |
| PVP-K90 (Polyvinyl | 10.4 | 0.0 | 0.0 | 0.0 |
| Pyrrolidone, ISP) | | | | |
| Glycerol | 10.4 | 10.8 | 19.0 | 2.9 |
| Water | 27.1 | 57.0 | 57.0 | 57.3 |
| Ethanol | 31.3 | 0 | 0 | 0 |
| Ketoprofen | saturated | saturated | saturated | saturated |

*Ingredients are noted as % by weight.

Each of the compositions of Examples 17-20 were studied for flux of ketoprofen, as shown in Table 16, as follows:

TABLE 16
Steady-state flux of Ketoprofen through hairless mouse skin
from various adhesive formulations at 35° C.

| | Average flux |
|---|---|
| Formulation | mcg/cm²/h* |
| Example 17 | 8 ± 3 |
| Example 18 | 21 ± 6 |
| Example 19 | 3 ± 1 |
| Example 20 | 1 ± 0.4 |
| *Skin flux measurements represent the mean and standard deviation of three determinations. Flux measurements reported were determined from the linear region of the cumulative amount versus time plots. The linear region was observed to be between 4-8 hours. | |
| # If experimental conditions allowed the steady state flux would extend beyond the 8 hours measured. | |

Regarding formulation described in Example 17, ethanol and water formed the volatile solvent system, while a 1:1 mixture of glycerol and PEG 400 formed the non-volatile solvent system. Through experimentation, it is determined that PEG 400 is a slightly better solvent than glycerol for ketoprofen, while glycerol is much more compatible with polyvinyl alcohol than polyethylene glycol (PEG 400). Thus, the non-volatile solvent system of glycerol and PEG 400 are used together to provide a non-volatile solvent system for the drug, while being reasonably compatible with polyvinyl alcohol. In additional detail with respect to the formulation in Example 17, polyvinyl alcohol and polyvinyl pyrrolidone act as the solidifying agents. Further, in this embodiment, glycerol and PEG 400 also serve as plasticizers in the adhesive formulation formed after the evaporation of the volatile solvents. Without the presence of glycerol and PEG 400, a solidified layer formed by polyvinyl alcohol and polyvinyl pyrrolidone alone would be rigid and non-stretchable.

Regarding the formulation of Example 18, the adhesive formation formed has similar physical properties as that of Example 17, though the transdermal flux across hairless mouse skin is higher. This suggests that the solidifying agent, 1:1 PVA:PVP-K-90 in Example 17 and pure polyvinyl alcohol in Example 19, have an impact on permeation.

The formulation in Example 19 delivers less ketoprofen than the formulations of Examples 17 or 18. The formulation of Example 20 delivers much less ketoprofen than the formulations in Examples 17 and 18. One possible reason for the reduced flux is believed to be the reduced permeation driving force caused by the high concentration of PEG 400 in the non-volatile solvent system, which resulted in too high of solubility for ketoprofen.

The only significant difference among the formulations in Examples 18, 19, and 20, respectively, is with respect to the non-volatile solvent system, or more specifically, the PEG 400:glycerol weight ratio. These results reflect the impact of the non-volatile solvent system on skin flux.

### Example 21

A stretchable adhesive formulation for transdermal delivery of ketoprofen (which is suitable for delivery via skin on joints and muscles) is prepared which includes saturated amount of ketoprofen in an excipient mixture (more ketoprofen than that can be dissolved in the excipient mixture) to form an adhesive formulation, some of which are prepared in accordance with embodiments of the present invention. The excipient mixture, which is a viscous and transparent fluid, is prepared using the ingredients as shown in Table 17.

TABLE 17

| | FORMULATIONS | | |
|---|---|---|---|
| Ingredients* | A | B | C |
| PVA (Celvol 502 MW 10,000) | 24.4 | | |
| PVA (Amresco MW 31,000-50,000) | | 24.4 | |
| PVA (Celvol 523 MW 125,000) | | | 41.7 |
| Water | 33.4 | 33.4 | 58.3 |
| Ethanol | 8.9 | 8.9 | |
| PG | 17.8 | 17.8 | |
| Glycerol | 11.1 | 11.1 | |
| Gantrez ES 425 | 4.4 | 4.4 | |

*Ingredients are noted in weight percent.

Formulations A and B are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.
   Preparation of the polyvinyl alcohol in water solution in Formulation C was not feasible for this molecular weight of polyvinyl alcohol because the polymer would not dissolve in water at the percentages noted. Formulation C demonstrates that the correct polymer molecular weight for polyvinyl alcohol is important to obtain the desired formulation properties.

Formulations A and B are placed on the skin of human volunteers. After a period of several hours, more than long enough for the volatile solvent to evaporate and for the solidified layer to form, the solidified layers were removed by the volunteers and the peelability properties were evaluated. In all instances the volunteers reported that formulation example A could not be removed in one or two pieces, but was removed in numerous small pieces. Formulation example B removed in one or two pieces. The brittle nature of formulation A is attributed to the lower molecular weight polyvinyl alcohol sample (Celvol 502). Low molecular weight polyvinyl alcohol does not possess the same cohesive strength as higher molecular weight polyvinyl alcohol material (Amresco) due to the reduced size of the polymer chain leading to a reduction in the degree of cross linking and physical interactions between individual polyvinyl alcohol polymer chains. The reduced polyvinyl alcohol chain interactions lead to a weakened solidified layer that is unable to withstand the mechanical forces the solidified layer is subjected to upon removal.

### Examples 22-23

A stretchable adhesive formulation for transdermal delivery of ketoprofen (which is suitable for delivery via skin on joints and muscles) was evaluated which includes a placebo excipient mixture which will form an adhesive formulation, some of which are prepared in accordance with embodiments of the present invention. The excipient mixture, which is a viscous and transparent fluid, is prepared using the ingredients as shown in Table 18.

TABLE 18

Examples

| Ingredients* | 22 | 23 |
|---|---|---|
| PVA (Amresco MW 31,000-50,000) | 20.41 | 21.28 |
| Water | 30.61 | 27.66 |
| Ethanol | 20.41 | 21.28 |
| PG | 20.41 | 21.28 |
| Glycerol | 6.12 | 6.38 |
| Gantrez S97 | 2.04 | 2.13 |

*Ingredients are noted in weight percent.

Peel formulations in Examples 22 and 23 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez S97 is added to the mixture.
The resulting solution is vigorously mixed for several minutes.

Formulations above were applied on the forearms of study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the peel from the skin or leaving residue behind. The time when this observation is made is defined as the drying time for the peel formulation. The results of the study are summarized in Table 19 below.

TABLE 19

| Example | Drying Time (min) |
|---|---|
| 22 | 7.0 |
| 23 | 6.5 |

The amount of water in the formulation did not significantly influence the time for the formulation to dry. However, it was noted during the study that the formulation was difficult to expel from the sample tube. After approximately 4 weeks after the formulation in Examples 22 and 23 were made the sample tubes were retrieved and were evaluated for ease of dispensing the formulation. It was noted that the formulation was impossible to expel from the tube. Interpolymer complexation between Gantrez S-97 and polyvinyl alcohol through electrostatic interactions, hydrophobic interactions, hydrogen bonding, or Van der Waals interactions is hypothesized to be the reason(s) for the observed thickening. Moreover, the extent of this interaction may be dependent on the stoichiometric ratio of the two polymers. This example sets forth embodiments where it is beneficial to select appropriate auxiliary solidifying agents.

### Examples 24-27

A stretchable adhesive formulation for transdermal delivery of ketoprofen (which is suitable for delivery via skin on joints and muscles) was evaluated which includes an excipient mixture which will form an adhesive formulation, some of which are prepared in accordance with embodiments of the present invention. The excipient mixture, which is a viscous and transparent fluid, is prepared using the ingredients as shown in Table 20.

TABLE 20

| | Examples | | | |
|---|---|---|---|---|
| Ingredients* | 24 | 25 | 26 | 27 |
| PVA (Amresco MW 31,000-50,000) | 22.1 | 24.4 | 22.1 | 21.1 |
| Water | 26.6 | 29.2 | 30.9 | 33.8 |
| Ethanol | 12.6 | 4.2 | 8.4 | 8.2 |
| Butanol | 0.4 | 0.5 | 0.4 | 0.4 |
| PG | 19.9 | 21.9 | 17.7 | 16.9 |
| Glycerol | 8.8 | 9.7 | 11 | 10.6 |
| Gantrez ES 425 | 4.6 | 5.1 | 4.4 | 4.0 |
| Ketoprofen | 5.0 | 5.0 | 5.1 | 5.0 |

*Ingredients are noted in weight percent.

Peel formulations in Examples 24-27 are prepared in the following manner:
PVA (solidifying agent) is dissolved in water.
The flux adequate non-volatile solvent (glycerol, PG) is mixed together with the solidifying agent/volatile solvent mixture.
Then ethanol, and Gantrez ES 425 is added to the mixture.
The resulting solution is vigorously mixed for several minutes.
After mixing, ketoprofen is added and the final mixture is vigorously mixed again for several minutes.

Formulations noted above were placed in laminate packaging tubes and stored at 25 C/60% RH and 40 C/75% RH conditions until pulled for testing. Physical testing was performed on each formulation. Examples 24-26 have been studied the longest and the resulting viscosity increase necessitated the desire to study the viscosity of Example 27. Table 21 summarizes the data generated on each formulation.

TABLE 21

| | Viscosity* | | | | | |
|---|---|---|---|---|---|---|
| Example | cPs | | | | | |
| Storage Cond. | T = 0 | 2 weeks | 4 weeks | 8 weeks | 12 weeks | 16 weeks |
| 24 | 96000 | 670000 | >2500000 | Not measured | | |
| 25 C./60% RH | | | | | | |
| 24 | 96000 | 500000 | 587500 | 2320000 | | |
| 40 C./75% RH | | | | | | |
| 25 | 168500 | 204500 | 251000 | >2500000 | | |
| 25 C./60% RH | | | | | | |
| 25 | 168500 | 215000 | 217500 | >2500000 | | |
| 40 C./75% RH | | | | | | |
| 26 | 23000 | — | 25000 | 36250 | 76250 | 57500 |
| 25 C./60% RH | | | | | | |
| 26 | 23000 | — | 31000 | 40000 | 243500 | 164500 |
| 40 C./75% RH | | | | | | |
| 27 | 11250 | 13750 | | | | |
| 25 C./60% RH | | | | | | |
| 27 | 11250 | 17500 | | | | |
| 40 C./75% RH | | | | | | |

*Viscosity measured using a RVDV 1+ viscometer at 0.5 rpm.

Examples 24 and 25 had the lowest water content of the four formulations and within 4 weeks of storage attained high viscosity values. The only difference between Examples 24 and 25 is the amount of ethanol in the formulations. It was hypothesized that reducing the level of ethanol may reduce the physical thickening of the formulation due to an incompatibility between the polyvinyl alcohol and ethanol. The viscosity data show that the higher ethanol formulation (formulation 1) had lower initial viscosity, but over the 4 weeks storage the viscosity of both Examples 24 and 25 attained viscosity values that were too high for a viable formulation. Another hypothesis for the formulation thickening is that polyvinyl alcohol is not compatible in high concentrations when dissolved in water. Additional formulations with higher water content were prepared to determine if an optimal water amount would keep the formulation from thickening up over time. Example 26 viscosity after 16 weeks has not reached the viscosity values of the initial viscosity values of Examples 24 and 25.

Placebo versions of the formulations above were applied on study volunteers and the drying time was assessed by placing a piece of cotton to the application site and then applying a 5 gram weight on the cotton. The cotton and weight was removed after 5 seconds. This procedure was started approximately 3-4 minutes after application and at 10 to 60 second intervals thereafter until the cotton was removed without lifting the peel or leaving residue behind. The results of the study are summarized in Table 22 below.

TABLE 22

| Formulation | Drying Time (min)* |
|---|---|
| 24 | 4 min 49 sec |
| 25 | 5 min 41 sec |
| 26 | 4 min 27 sec |
| 27 | 5 min 1 sec |

*average dry time value from 12 study subjects.

The presence of ethanol as a second volatile solvent appears to significantly reduce the time to dry. In data not shown a local anesthetic formulation containing only water as the volatile solvent and a ratio of water to polyvinyl alcohol of 2:1 has a drying time of >15 minutes. Optimizing the ratio and the presence of an additional volatile solvent in formulations containing water significantly reduce the drying time. It is hypothesized that the additional volatile solvent, in this case ethanol, will hydrogen bond with the water and water will escape with the ethanol when evaporating off the skin thereby forming a solidified peel. This example illustrates that even relatively small differences in the water content and/or water:ethanol ratio can have significant impact on the long term stability and viability of the product.

### Examples 28-29

A stretchable adhesive formulation for transdermal delivery of ketoprofen (which is suitable for delivery via skin for treating inflammation or pain of joints and muscles) is prepared which includes ketoprofen in an excipient mixture to form an adhesive formulation, some of which is prepared in accordance with embodiments of the present invention. The peel formulation is prepared from the ingredients as shown in Table 23.

TABLE 23
Ketoprofen solidifying formulation components

| | Example | Example |
|---|---|---|
| Ingredients* | 28 | 29 |
| PVA | 22.1 | 18.9 |
| Water | 30.9 | 37.9 |
| Fumed Silicia | | 3.0 |
| Glycerol | 11.1 | 9.5 |
| Propylene glycol | 17.7 | 15.2 |
| Gantrez ES-425 | 4.4 | 3.8 |
| Ethanol | 8.8 | 7.6 |
| Ketoprofen | 5.0 | 4.2 |

*Ingredients are noted as weight percent.

TABLE 24
Steady-state flux of ketoprofen through hairless mouse skin
from an adhesive solidifying formulations at 35° C.

| | Average flux |
|---|---|
| Formulation | mcg/cm²/h* |
| Example 28 | 25 ± 6 |
| Example 29 | 27 ± 2 |

*Skin flux measurements represent the mean and standard deviation of three determinations. Flux measurements reported were determined from the linear region of the cumulative amount versus time plots. The linear region was observed to be between 4-8 hours. If experimental conditions allowed the steady state flux would extend beyond the 8 hours measured.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A formulation for dermal delivery of a drug, comprising
a) a drug;
b) polyvinyl alcohol; and
c) a solvent vehicle, comprising
i) a volatile solvent system including water and an alcohol solvent;
ii) a non-volatile solvent system, including at least one non-volatile solvent that is less volatile than water,
wherein the water to polyvinyl alcohol weight ratio is from about 4:1 to about 1:1, and the water to alcohol solvent weight ratio is from about 0.33:1 to about 6:.

2. A formulation as in claim 1, wherein the non-volatile solvent system facilitates dermal delivery of the drug at a therapeutically effective rate over a sustained period of time.

3. A formulation as in claim 1, wherein the weight ratio of the non-volatile solvent system to polyvinyl alcohol is between 0.5:1 to 2:1.

4. A formulation as in claim 1, wherein the weight average molecular weight of the polyvinyl alcohol is more than 10,000.

5. A formulation as in claim 1, wherein the weight average molecular weight of the polyvinyl alcohol is less than 125,000.

6. A formulation as in claim 1, wherein the alcohol solvent includes at least one solvent selected from the group consisting of ethanol, isopropanol, and combinations thereof.

7. A formulation as in claim 1, wherein the non-volatile solvent system includes a member selected from the group of glycerol, propylene glycol, isostearic acid, sorbitan monolaurate, oleic acid, olelyl alcohol, poly ethylene glycol, and combinations thereof.

8. A formulation as in claim 1, wherein the drug includes a member selected from the group acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, and combinations thereof.

9. A formulation as in claim 1, wherein the drug includes at least one member selected from the group consisting of amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, erythromycin, clindamycin, tetracycline, bacitracin, neomycin, mupirocin, polymyxin B, quinolones, ciproflaxin, bupivacaine, alpha-2 agonists, clonidine, amitriptyline, carbamazepine, alprazolam, ketamine, ketanserin, betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, piroxicam, diclofenac, indomethacin, imiquimod, rosiquimod, salicylic acid, alpha hydroxy acids, sulfur, rescorcinol, urea, benzoyl peroxide, allantoin, tretinoin, trichloroacetic acid, lactic acid, benzoic acid, progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, testosterone, methyl testosterone, oxandrolone, androstenedione, dihydrotestosterone, estradiol, ethniyl estradiol, estiol, estrone, conjugated estrogens, esterified estrogens, estropipate, and combinations thereof.

10. A formulation as in claim 1, wherein the drug includes multiple pharmaceutically active agents.

11. A formulation as in claim 1, wherein the water to polyvinyl alcohol weight ratio is from about 3:1 to about 1.2:1.

12. A formulation as in claim 1, wherein the water to polyvinyl alcohol weight ratio is from about 2:1 to about 1.4:1.

13. A formulation as in claim 1, wherein the water to alcohol solvent weight ratio is from about 0.75:1 to about 6:1.

14. A formulation as in claim 1, wherein the water to alcohol solvent weight ratio is from about 0.75:1 to about 2:1.

15. A formulation as in claim 1, wherein the formulation further comprises a second solidifying agent in addition to the polyvinyl alcohol.

16. A formulation as in claim 1, wherein at least one non-volatile solvent of the non-volatile solvent system acts as a plasticizer for the polyvinyl alcohol.

17. A formulation as in claim 1, wherein the formulation further comprises an additional agent which is added to increase adhesion of the solidified formulation layer to the skin surface.

18. A formulation as in claim 17, wherein the additional agent includes a member selected from the group consisting of copolymers of methylvinyl ether and maleic anhydride, polyethylene glycol and polyvinyl pyrrolidone, gelatin, low molecular weight polyisobutylene rubber, copolymer of acrylsan alkyl/octylacrylamido, aliphatic resins, aromatic resins, and combinations thereof.

19. A formulation as in claim 1, wherein the volatile solvent system comprises at least one additional solvent that is more volatile than water, and is miscible with water.

20. A formulation as in claim 1, wherein the formulation has an initial viscosity prior to skin application from about 100 cP to about 3,000,000 cP.

21. A formulation as in claim 1, wherein the formulation has an initial viscosity prior to skin application from about 1,000 cP to about 1,000,000 cP.

22. A formulation as in claim 1, wherein the formulation, after applied to a skin surface as a layer, forms a coherent solidified layer after evaporation of at least some of the volatile solvent system.

23. A formulation as in claim 1, wherein the formulation has a viscosity suitable for application and adhesion to a skin surface prior to evaporation of the volatile solvent system, wherein the formulation applied to the skin surface as a layer forms a solidified layer after at least partial evaporation of the volatile solvent system, and wherein the drug continues to be delivered after the volatile solvent system has at least substantially evaporated.

24. A formulation as in claim 23, wherein the solidified layer is peelable from the skin surface as a coherent solid after the water and the alcohol solvent has at least partially evaporated.

25. A formulation as in claim 1, wherein the dermal delivery is transdermal delivery.

26. A formulation as in claim 1, wherein the volatile solvent system comprises a volatile solvent whose boiling point is below 20° C.

27. A formulation as in claim 26, wherein the volatile solvent with the boiling point below 20° C. is completely dissolved in the formulation.

28. A formulation as in claim 26, wherein the volatile solvent with the boiling point below 20° C. is included in the formulation as a propellant for pressurized spray-on application.

29. A formulation as in claim 26, wherein the volatile solvent with the boiling point below 20° C. is a hydrofluorocarbon.

30. The formulation as in claim 26, wherein the at least one solvent whose boiling point is below 20° C. is selected from the group consisting of dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof.

31. The formulation as in claim 1, wherein the solidified layer is formulated to transdermally deliver the drug.

32. A method of dermally delivering a drug, comprising:
a) applying a formulation to a skin surface of a subject, the formulation, comprising:
i) a drug;
ii) a polyvinyl alcohol; and
iii) a solvent vehicle, comprising
a volatile solvent system including water and an alcohol solvent, and
a non-volatile solvent system including at least one non-volatile solvent that is less volatile than water, wherein the non-volatile solvent system facilitates dermal delivery of the drug at a therapeutically effective rate over a sustained period of time;
wherein the water to polyvinyl alcohol weight ratio is from about 4:1 to about 1:1, and the water to alcohol solvent weight ratio is from about 0.33:1 to about 6:1;
b) solidifying the formulation to form a solidified layer on the skin surface by at least partial evaporation of the volatile solvent system; and
c) dermally delivering the drug from the solidified layer to the subject at therapeutically effective rates over a sustained period of time.

33. A method as in claim 32, wherein the alcohol solvent includes a member selected from the group consisting of ethanol, propanol, isopropanol, and combinations thereof.

34. A method as in claim 32, wherein the step of applying includes applying the formulation at a thickness from about 0.01 mm to about 3 mm.

35. A method as in claim 34, wherein the thickness is from about 0.05 mm to about 1 mm.

36. A method as in claim 32, wherein the weight ratio of the non-volatile solvent system to polyvinyl alcohol is between 0.5:1 to 2:1.

37. A method as in claim 32, wherein the weight average molecular weight of the polyvinyl alcohol is between about 10,000 and 125,000.

38. A method as in claim 32, wherein the non-volatile solvent system includes at least one member selected from the group of glycerol, propylene glycol, isostearic acid, sorbitan monolaurate, oleic acid, olelyl alcohol, poly ethylene glycol, and combinations thereof.

39. A method as in claim 32, wherein the drug includes a member selected from the group acyclovir, econazole, miconazole, terbinafine, lidocaine, bupivacaine, ropivacaine, and tetracaine, amitriptyline, ketanserin, betamethasone dipropionate, triamcinolone acetonide, clindamycin, benzoyl peroxide, tretinoin, isotretinoin, clobetasol propionate, halobetasol propionate, ketoprofen, piroxicam, diclofenac, indomethacin, imiquimod, salicylic acid, benzoic acid, and combinations thereof.

40. A method as in claim 32, wherein the drug includes at least one member selected from the group consisting of amorolfine, butenafine, naftifine, terbinafine, fluconazole, itraconazole, ketoconazole, posaconazole, ravuconazole, voriconazole, clotrimazole, butoconazole, econazole, miconazole, oxiconazole, sulconazole, terconazole, tioconazole, caspofungin, micafungin, anidulafingin, amphotericin B, AmB, nystatin, pimaricin, griseofulvin, ciclopirox olamine, haloprogin, tolnaftate, undecylenate, penciclovir, famciclovir, valacyclovir, behenyl alcohol, trifluridine, idoxuridine, cidofovir, gancyclovir, podofilox, podophyllotoxin, ribavirin, abacavir, delavirdine, didanosine, efavirenz, lamivudine, nevirapine, stavudine, zalcitabine, zidovudine, amprenavir, indinavir, nelfinavir, ritonavir, saquinavir, amantadine, interferon, oseltamivir, ribavirin, rimantadine, zanamivir, erythromycin, clindamycin, tetracycline, bacitracin, neomycin, mupirocin, polymyxin B, quinolones, ciproflaxin, bupivacaine, alpha-2 agonists, clonidine, amitriptyline, carbamazepine, alprazolam, ketamine, ketanserin, betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, tacrolimus, picrolimus, tazarotene, isotretinoin, cyclosporin, anthralin, vitamin D3, cholecalciferol, calcitriol, calcipotriol, tacalcitol, calcipotriene, piroxicam, diclofenac, indomethacin, imiquimod, rosiquimod, salicylic acid, alpha hydroxy acids, sulfur, rescorcinol, urea, benzoyl peroxide, allantoin, tretinoin, trichloroacetic acid, lactic acid, benzoic acid, progesterone, norethindrone, norethindroneacetate, desogestrel, drospirenone, ethynodiol diacetate, norelgestromin, norgestimate, levonorgestrel, dl-norgestrel, cyproterone acetate, dydrogesterone, medroxyprogesterone acetate, chlormadinone acetate, megestrol, promegestone, norethisterone, lynestrenol, gestodene, tibolene, testosterone, methyl testosterone, oxandrolone, androstenedione, dihydrotestosterone, estradiol, ethniyl estradiol, estiol, estrone, conjugated estrogens, esterified estrogens, estropipate, and combinations thereof.

41. A method as in claim 32, wherein the water to polyvinyl alcohol weight ratio is from about 3:1 to about 1.4:1.

42. A method as in claim 32, wherein the water to alcohol solvent weight ratio is from about 0.33:1 to about 6:1.

43. A method as in claim 32, wherein the non-volatile solvent system is capable of acting as a plasticizer for polyvinyl alcohol.

44. A method as in claim 32, wherein the formulation has an initial viscosity prior to skin application from about 100 cP to about 3,000,000 cP.

45. A method as in claim 32, wherein the formulation has an initial viscosity prior to skin application from about 1,000 cP to about 1,000,000 cP.

46. A method as in claim 32, wherein the solidified layer is left on the skin surface for at least about 6 hours.

47. A solidified layer for dermally delivering a drug, comprising:
a) a drug;
b) a non-volatile solvent system including at least one non-volatile solvent, wherein the non-volatile solvent system is capable of facilitating the delivery of the drug at therapeutically effective rates over a sustained period of time; and
c) a polyvinyl alcohol,
wherein the solidified layer is capable of adhering to a skin surface to which the layer for at least two hours.

48. A solidified layer as in claim 47, which is coherent, flexible, and continuous.

49. A solidified layer as in claim 47, which is a soft, coherent solid that is peelable from a skin surface as a single piece or as only a few large pieces relative to the application size.

50. A solidified layer as in claim 47, wherein the weight ratio of the non-volatile solvent system to the solidifying agent is from about 0.5:1 to about 2:1.

51. A solidified layer as in claim 47, wherein the non-volatile solvent system comprises at least one solvent selected from the group consisting of propylene glycol, glycerol, isostearic acid, oleic acid, and combinations thereof.

52. A solidified layer as in claim 47, wherein the solidified layer can be stretched in at least one direction by 5% without separation from the skin surface.

53. A solidified layer as in claim 47, wherein the non-volatile solvent system acts as a plasticizer for the polyvinyl alcohol.

54. A solidified layer as in claim 47, wherein solidified layer is sufficiently adhesive and flexible to remain substantially intact on a skin surface adjacent to a joint or muscle group where regular skin stretching occurs.

55. A solidified layer as in claim 47, wherein the weight ratio of the non-volatile solvent system to the polyvinyl alcohol is from about 0.5:1 to about 2:1.

56. A solidified layer as in claim 47, wherein the solidified layer can be removed by washing.

57. A solidified layer as in claim 47, wherein the solidified layer substantially devoid of water and solvents more volatile than water in that it contains no more than 10 wt % of water and solvents more volatile than water, and wherein the solidified layer dermally delivers the drug, even in the substantial absence of the water and the solvents more volatile than water.

58. A solidified layer as in claim 57, wherein the solidified layer contains no more than 5 wt % of water and solvents more volatile than water.

59. A solidified layer as in claim 47, wherein the solidified layer is flux-enabling for the drug.
